(19) United States
(12) Patent Application Publication
(10) Pub. No.: US 2022/0026389 A1
(43) Pub. Date: Jan. 27, 2022

(54) ORGANIC THIN FILM TRANSISTOR GAS SENSOR SYSTEM WITH MONOLAYER AS BLOCKING LAYER ON SOURCE/DRAIN ELECTRODES

(71) Applicant: Sumitomo Chemical Co., Ltd., Tokyo (JP)

(72) Inventors: Pascal Cachelin, Cambridgeshire (GB); Nicholas Dartnell, Cambridgeshire (GB); Daniel Tobjork, Cambridgeshire (GB)

(73) Assignee: Sumitomo Chemical Co., Ltd., Tokyo (JP)

(21) Appl. No.: 17/424,488

(22) PCT Filed: Jan. 21, 2020
(86) PCT No.: PCT/GB2020/050125

§ 371 Date: Jul. 20, 2021

(30) Foreign Application Priority Data
Jan. 21, 2019 (GB) 1900834.1
Feb. 20, 2019 (GB) 1902308.4

(51) Int. Cl. G01N 27/414; H01L 51/05; G01N 33/00; G01N 33/02

(52) U.S. Cl. CPC G01N 27/4141; G01N 33/025; G01N 33/0047; H01L 51/0566

(57) ABSTRACT

A gas sensor system (100) for detecting conjugated hydrocarbons and/or esters in an environment. The gas sensor system includes two organic thin film transistor (OTFT) gas sensors (200, 300). The first OTFT gas sensor (200) allows for interaction of the conjugated hydrocarbon with the gas sensor's source and drain electrodes, and the second OTFT gas sensor (300) blocks the conjugated hydrocarbon from interacting with the gas sensor's source and drain electrodes. In the second gas sensor, a monolayer comprising 4-aminobenzenethiol and 4-fluorobenzenethiol may cover the source and drain electrodes preventing the conjugated hydrocarbon from interacting with the source and drain electrodes. The gas sensor system may be used to monitor a volatile conjugated hydrocarbon and/or an ester produced by fruit.

### BACKGROUND

Embodiments of the present disclosure relate to organic thin film transistor gas sensors. More particularly, but not by way of limitation, embodiments of the present disclosure relate to organic thin film gas sensors and gas sensor systems configured to detect conjugated hydrocarbons, e.g. styrene, and/or esters, e.g. butyl acetate.

Thin film transistors (TFTs) have been previously used as gas sensors. For example, such use of thin film transistors as gas sensors is described in Feng et al., Unencapsulated Air-stable Organic Field Effect Transistor by All Solution Processes for Low Power Vapor Sensing, SCIENTIFIC REPORTS 6:20671 DOI: 10.1038/srep20671 and Besar et al., Printable Ammonia Sensor Based on Organic Field Effect Transistor, ORGANIC ELECTRONICS, Vol. 15, Issue 11, pp. 3221-3230 (November 2014). In thin film transistor gas sensors, a semiconducting layer is in electrical contact with source and drain electrodes and a gate dielectric is disposed between the semiconducting layer and a gate electrode. Interaction of a target gas with the TFT gas sensor may alter the drain current of the TFT gas sensor.

Zhang et al., Organic Field-Effect Transistors-Based Gas Sensors, CHEM. SOC. REV., 44, 2087-2107 (2015) discloses gas discrimination systems having sensory arrays of organic field effect transistors.

Volatile organic compounds may be released during decay of food, for example as described in Phan, N T., Kim, K H., Jeon, E C. et al., Analysis of Volatile Organic Compounds Released During Food Decaying Processes, ENVIRON MONIT. ASSESS. 184: 1683. https://doi.org/10.1007/s10661-011-2070-2 (2012).

### SUMMARY

Volatile conjugated hydrocarbons such as styrene can be present in certain gaseous environments. For example, styrene may be produced by diseased or rotting food, e.g. diseased or rotting fruit, e.g. apples. Detection of the presence and/or concentration of styrene may therefore enable detection and management of rotting food. However, the present inventors have found that one or more other volatile organic compounds may be present in such environments, for example butyl acetate, which may be produced by both healthy and diseased or rotting fruit, making it difficult to differentiate the presence of styrene from other volatile organic compounds.

The present inventors have found that organic thin film transistors (OTFTs) may be used to detect the presence or concentration of volatile organic compounds in an environment, such as conjugated hydrocarbons, e.g. hydrocarbons containing a conjugated alkene group such as styrene and farnesene, and esters, e.g. butyl acetate. Further, the present inventors have found that sensitivity of an organic thin film transistor gas sensor to a given gas may be adjusted, allowing for differentiation between different gases in an environment by use of different organic thin film transistor gas sensors having different gas sensitivities.

In some embodiments of the present disclosure, a gas sensor system may contain two or more different OTFTs. The different OTFTs of the gas sensor system may be configured to detect different gases. For example, the present inventors have found that inhibiting the ability of a conjugated hydrocarbon, e.g. styrene or farnesene, to reach a surface of the source and drain electrodes of an OTFT gas sensor may reduce sensitivity of the OTFT gas sensor to the conjugated hydrocarbon.

Accordingly, in some embodiments there is provided a gas sensor system configured to detect a conjugated hydrocarbon in an environment. According to some embodiments of the present disclosure, the gas sensor system may contain first and second OTFT gas sensors. In such embodiments, each of the first and second OTFT gas sensors may comprise source and drain electrodes in electrical contact with an organic semiconductor layer, a gate electrode, and a gate dielectric between the organic semiconductor layer and the gate electrode. In some embodiments of the present disclosure, the first OTFT gas sensor may be configured to provide for gas communication of the conjugated hydrocarbon in the environment with the source and drain electrodes. The second OTFT gas sensor may be configured to block gas communication of the conjugated hydrocarbon in the environment with the source and drain electrodes. By measuring the response of the two OTFT gas sensors to the environment, presence and/or concentration of the conjugated hydrocarbon may be determined.

In some embodiments, there is provided a gas sensor system containing at least two different organic thin film transistor (OTFT) gas sensors. Each of the OTFT gas sensors may have source and drain electrodes in electrical contact with an organic semiconductor layer; a gate electrode; and a gate dielectric between the organic semiconductor layer and the gate electrode. The organic semiconducting layer of one of the OTFT gas sensors may contain an amorphous organic semiconductor and the organic semiconducting layer of another of the OTFT gas sensors may contain a crystalline organic semiconductor. The use of the different organic semiconductor materials provides that the gas sensors respond differently to the conjugated hydrocarbon, for example one of the gas sensors may have reduced/no sensitivity to the conjugated hydrocarbon, allowing for discrimination between different volatiles in the environment, such as volatiles produced by fruit and/or rotting fruit.

In some embodiments, there is provided a gas sensor system containing at least one top-gate organic thin film transistor gas sensor and at least one bottom gate organic thin film transistor.

In some embodiments, there is provided a gas sensor system comprising at least two different organic thin film transistor (OTFT) gas sensors, each of the OTFT gas sensors comprising source and drain electrodes in electrical contact with an organic semiconductor layer; a gate electrode; and a gate dielectric between the organic semiconductor layer and the gate electrode, wherein the organic semiconducting layer is the only semiconducting layer of one of the OTFT gas sensors and wherein another OTFT gas sensor comprises a second semiconducting layer.

In some embodiments there is provided an OTFT gas sensor configured to detect a conjugated hydrocarbon in an environment. The OTFT gas sensor may have source and drain electrodes in electrical contact with an organic semiconductor layer; a gate electrode; and a gate dielectric between the organic semiconductor layer and the gate electrode. The OTFT gas sensor may be configured to provide for gas communication of the conjugated hydrocarbon in the environment with the source and drain electrodes thereof.

In some embodiments there is provided a method of identifying the presence and/or concentration of a conjugated hydrocarbon in an environment. The method may include measurement of a response of an OTFT gas sensor as described herein to the environment, and determining from the measured response if the conjugated hydrocarbon is present and/or determining a concentration of the conjugated hydrocarbon.

In some embodiments there is provided an OTFT gas sensor configured to detect an ester in an environment. The OTFT gas sensor may have source and drain electrodes in electrical contact with an organic semiconductor layer; a gate electrode; and a gate dielectric between the organic semiconductor layer and the gate electrode. The OTFT gas sensor configured to detect an ester may be configured to block gas communication of gases in the environment with the source and drain electrodes thereof.

### DESCRIPTION OF THE DRAWINGS

The disclosed technology and accompanying figures describe some implementations of the disclosed technology.

FIG. 1 illustrates a gas sensor system, according to some embodiments of the present disclosure;

FIG. 2 illustrates a bottom gate, bottom contact OTFT gas sensor, according to some embodiments of the present disclosure;

FIG. 3 illustrates a bottom gate, top contact OTFT gas sensor, according to some embodiments of the present disclosure;

FIG. 4 illustrates a top gate, bottom contact OTFT gas sensor, according to some embodiments of the present disclosure;

FIG. 5 illustrates a top gate, top contact OTFT gas sensor, according to some embodiments of the present disclosure;

FIG. 6 illustrates a bottom gate, bottom contact OTFT gas sensor, according to some embodiments of the present disclosure, having a blocking layer disposed on a surface of the source and drain electrodes;

FIG. 7 is a graph of drain current versus time upon exposure to butyl acetate of two different OTFT gas sensors, according to some embodiments of the present disclosure; and

FIG. 8 is a graph of drain current versus time upon exposure to styrene of the two different OTFT gas sensors of FIG. 7.

The drawings are not drawn to scale and have various viewpoints and perspectives. The drawings are some implementations and examples. Additionally, some components and/or operations may be separated into different blocks or combined into a single block for the purposes of discussion of some of the embodiments of the disclosed technology. Moreover, while the technology is amenable to various modifications and alternative forms, specific embodiments have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof means any connection or coupling, either direct or indirect, between two or more elements; the coupling or connection between the elements can be physical, logical, electromagnetic, or a combination thereof. Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list.

As used herein, by a material “over” a layer is meant that the material is in direct contact with the layer or is spaced apart therefrom by one or more intervening layers.

As used herein, by a material “on” a layer is meant that the material is in direct contact with that layer.

A layer “between” two other layers as described herein may be in direct contact with each of the two layers it is between or may be spaced apart from one or both of the two other layers by one or more intervening layers.

The teachings of the technology provided herein can be applied to other systems, not necessarily the system described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. Details of the system may vary considerably in its specific implementation, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms. For example, while some aspect of the technology may be recited as a computer-readable medium claim, other aspects may likewise be embodied as a computer-readable medium claim, or in other forms, such as being embodied in a means-plus-function claim.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

The techniques introduced here can be embodied as special-purpose hardware (e.g., circuitry), as programmable circuitry appropriately programmed with software and/or firmware, or as a combination of special-purpose and programmable circuitry. Hence, embodiments may include a machine-readable medium having stored thereon instructions which may be used to program a computer (or other electronic devices) to perform a process. The machine-readable medium may include, but is not limited to, floppy diskettes, optical disks, compact disc read-only memories (CD-ROMs), magneto-optical disks, ROMs, random access memories (RAMs), erasable programmable read-only memories (EPROMs), electrically erasable programmable read-only memories (EEPROMs), magnetic or optical cards, flash memory, or other type of media/machine-readable medium suitable for storing electronic instructions. The machine-readable medium includes non-transitory medium, where non-transitory excludes propagation signals. For example, a processor can be connected to a non-transitory computer-readable medium that stores instructions for executing instructions by the processor.

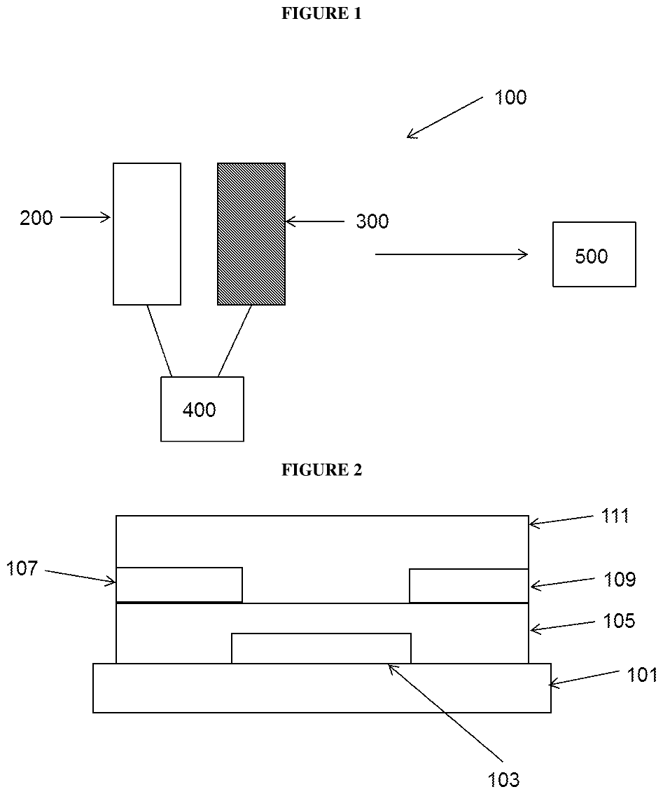

FIG. 1 is a schematic illustration of a gas sensor system 100 according to some embodiments of the present disclosure. The gas sensor system comprises a first OTFT gas sensor 200 and a second OTFT gas sensor 300 which is different from the first OTFT gas sensor. The first gas sensor may be configured to detect a first gas. The second gas sensor may be configured to detect a second gas which is different from the first gas.

In some embodiments, the sensors 200, 300 are connected to a measurement unit 400 configured to measure a response of the sensors upon exposure to a gaseous environment. The response may be a change in drain current. FIG. 1 illustrates sensors connected to the same measurement unit 400. In other embodiments, different OTFT gas sensors may be connected to different measurement units.

The or each measurement unit 400 may be in wired or wireless communication with processor unit 500 configured to determine, from received measurements of the first and second OTFT gas sensors or a derivative thereof, a presence, a concentration, and/or a change in concentration, of the first gas and/or the second gas.

The gas sensor system may be in wired or wireless communication with a user interface providing information on the presence, concentration and/or change in concentration of the first and second gases in the environment.

Each of the OTFT gas sensors of the gas sensor system may be supported on a common substrate and/or contained in a common housing.

In use, each OTFT gas sensor may be connected to a common power source, or two or more of the OTFT gas sensors may be powered by different power sources.

In use, power to all of the OTFT gas sensors of the gas sensor system may be controlled by a single switch or power to two or more of the OTFT gas sensors may be controlled by different switches.

FIG. 1 illustrates a gas sensor system having only two different OTFT gas sensors. In other embodiments, a gas sensor system as described anywhere herein may comprise three or more different OTFT gas sensors.

FIG. 1 illustrates a gas sensor system having only one first OTFT gas sensor and only one second OTFT gas sensor. In other embodiments, a gas sensor system may comprise a plurality of first OTFT gas sensors and/or a plurality of second OTFT gas sensors.

The gas sensor system may comprise one or more control OTFT gas sensors to provide a baseline for measurements of the first and second OTFT gas sensors to take into account variables such as one or more of humidity, temperature, pressure, variation of sensor parameter measurements over time (such as variation of OTFT sensor drain current over time), and gases other than a target gas or target gases in the atmosphere. One or more control OTFT gas sensors may be isolated from the atmosphere, for example by encapsulation of the or each OFT control sensor, to provide a baseline measurement other than gases in the atmosphere.

In some embodiments, the first OTFT gas sensor has greater sensitivity, optionally at least 2 or 3 times greater sensitivity, to the first gas than the second OTFT gas sensor. In some embodiments, the first gas is a conjugated hydrocarbon, optionally a conjugated alkene hydrocarbon. The conjugated alkene hydrocarbon may comprise a double bond conjugated to an aromatic group, e.g. a double bond conjugated to benzene, such as styrene. The conjugated alkene hydrocarbon may comprise two or more alkene groups conjugated together, e.g. farnesene.

In some embodiments, the second OTFT gas sensor has greater sensitivity, optionally at least 2 or 3 times greater sensitivity, to the second gas than the first OTFT gas sensor. In some embodiments, the second gas is an ester, optionally a C₁₋₁₀ alkyl acetate and/or a C₁₋₁₀ alkanoate ester, optionally butyl acetate.

By “sensitivity” of an OTFT gas sensor to a gas as used herein is meant a maximum percentage change in a measured parameter of the OTFT gas sensor, preferably drain current, upon exposure of the OTFT gas sensor to a given concentration of the gas.

Optionally, sensitivity to a conjugated hydrocarbon, e.g. styrene, is as measured at a concentration of 0.1 ppm of the conjugated hydrocarbon.

Optionally, sensitivity to an ester, e.g. butyl acetate, is as measured at a concentration of 4000 ppm of the ester.

In some embodiments, the different sensitivities of the first and second OTFT gas sensors to a gas are provided by providing one or more of the following differences between the first and second OTFT gas sensors:
Different gate arrangements of the first and second OTFT gas sensors, e.g. top gate for one of the first and second OTFT gas sensors and bottom gate for the other OTFT gas sensor.
Different OTFT device types, e.g. top gate for one of the first and second OTFT gas sensors and bottom gate for the other OTFT gas sensor.
Different source and drain electrodes for the first and second OTFT gas sensors.
Different interfaces of the first and second OTFT gas sensors between the organic semiconducting layer and the source and drain electrodes of the first and second OTFT gas sensors.
Different organic semiconductors for the first and second OTFT gas sensors, e.g. a crystalline organic semiconductor for one of the first and second OTFT gas sensors and amorphous for the other OTFT gas sensor.
Different numbers of semiconducting layers, e.g. an organic semiconducting layer as the only semiconducting layer for one of the first and second OTFT gas sensors and at least one further semiconducting layer for the other OTFT gas sensor.
Different thicknesses of the organic semiconducting layers of the first and second OTFT gas sensors.

The first and second OTFT gas sensors may each independently be bottom-gate or top-gate OTFTs.

FIG. 2 is a schematic illustration of a bottom contact, bottom gate OTFT gas sensor in a gas sensor system as described herein. The bottom contact bottom gate OTFT comprises a gate electrode 103 over a substrate 101, e.g. a glass or plastic substrate; source and drain electrodes 107, 109; a dielectric layer 105 between the gate electrode and the source and drain electrodes; and an organic semiconductor layer 111 in electrical contact with the source and drain electrodes. The organic semiconductor layer 111 may at least partially or completely cover the source and drain electrodes. The organic semiconductor layer 111 may be in direct contact with a surface of each of the source and drain electrodes.

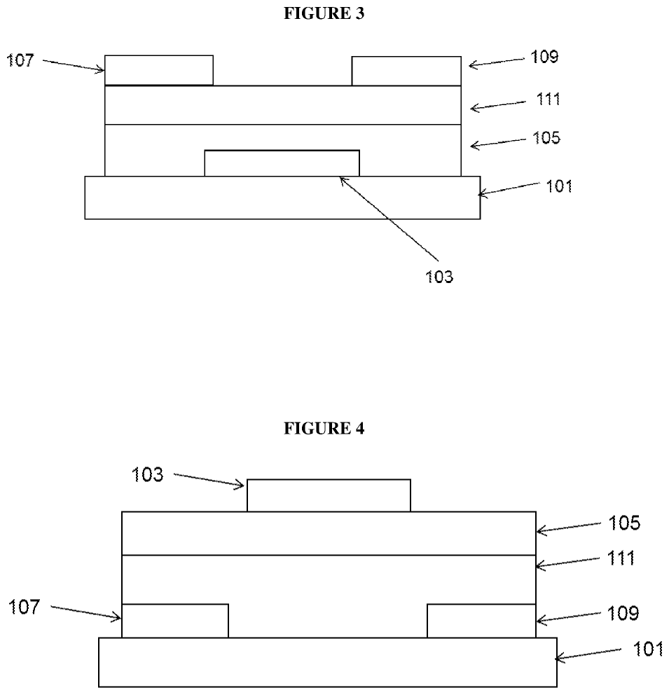

FIG. 3 is a schematic illustration of a top-contact bottom gate OTFT gas sensor in a gas sensor system as described herein. The top-contact bottom gate OTFT is as described with reference to FIG. 2 except that the organic semiconductor layer 111 is between the dielectric layer 105 and the source and drain electrodes 107, 109.

FIG. 4 is a schematic illustration of a top gate bottom contact OTFT gas sensor in a gas sensor system as described herein. The top gate bottom contact OTFT comprises source and drain electrodes 107, 109 disposed over a substrate; an organic semiconductor layer 111 in electrical contact with the source and drain electrodes; and a dielectric layer 105 between the gate electrode 103 and the organic semiconductor layer. The organic semiconducting layer is disposed between the source and drain electrodes and the dielectric layer.

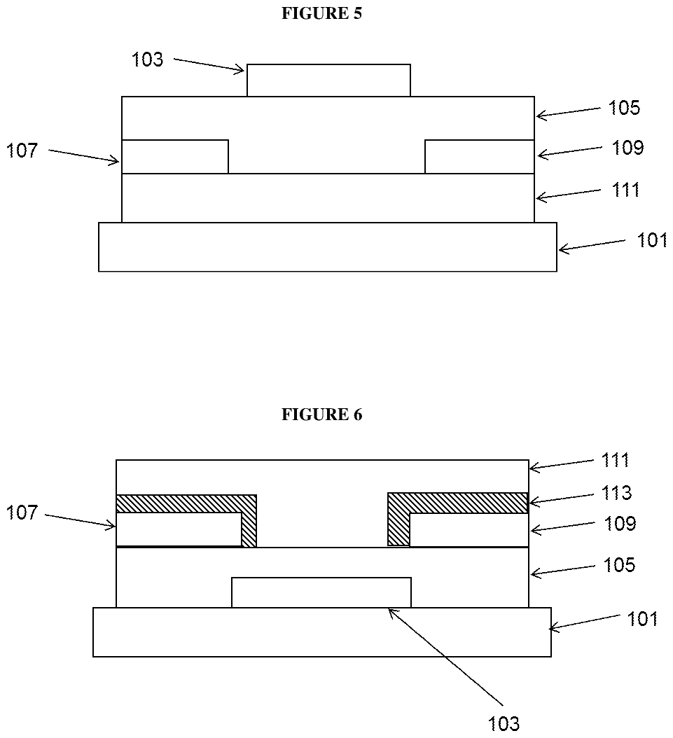

FIG. 5 is a schematic illustration of a top gate top contact OTFT gas sensor in a gas sensor system as described herein. The top gate OTFT is as described with reference to FIG. 4 except that the organic semiconducting layer is disposed between the substrate and the source and drain electrodes.

One of the first and second OTFT gas sensors, for example as described with reference to any one of FIGS. 2-5, may be modified so as to increase or decrease it sensitivity to a target gas relative to the other of the first and second OTFT gas sensors.

In some embodiments, a blocking layer may be disposed on a surface of the source and drain electrodes and between the source and drain electrodes and the organic semiconducting layer, for example as illustrated in FIG. 5.

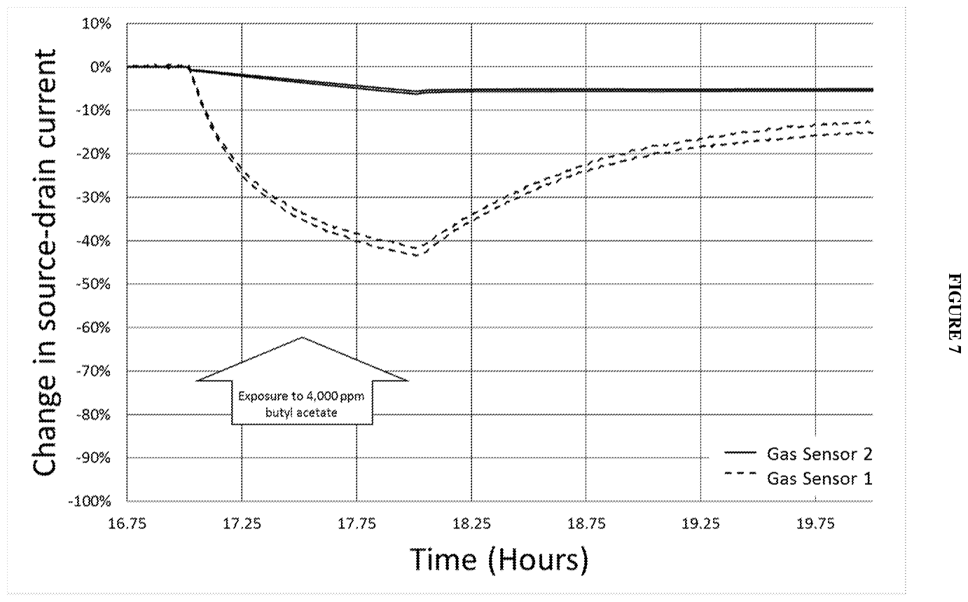

FIG. 6 is a schematic illustration of a bottom contact, bottom gate OTFT as described with reference to FIG. 2 except that a blocking layer 113 is disposed on surface of the source and drain electrodes 107, 109 and between the source and drain electrodes and the organic semiconducting layer. The blocking layer may decrease the sensitivity of the OTFT gas sensor, e.g. by inhibiting a target gas from binding to the source and drain electrodes. The blocking layer may alter the work function of the source and drain electrodes at the surface it is disposed on.

It will be understood that a blocking layer may be disposed on a surface of the source and drain electrodes and between the source and drain electrodes and the organic semiconductor layer of a top contact, bottom gate TFT, for example as described with reference to FIG. 3, or a top gate OTFT, for example as described with reference to FIG. 4

In some embodiments, the first OTFT gas sensor and/or the second OTFT gas sensor comprises a second semiconducting layer adjacent to the organic semiconducting layer. Such an OTFT gas sensor may be as described in any one of FIGS. 2-5, the OTFT gas sensor further comprising the second semiconducting layer adjacent to the organic semiconducting layer.

In some embodiments, the second semiconducting layer is disposed between the source and drain electrodes and the organic semiconducting layer.

In some embodiments, the organic semiconducting layer is disposed between the source and drain electrodes and the second semiconducting layer.

In some embodiments, gas in an atmosphere that a top gate OTFT gas sensor is exposed to is in gas communication with the organic semiconducting layer and/or the source and drain electrodes. In some embodiments, the dielectric layer of a top-gate OTFT gas sensor is a gas-permeable material, preferably an organic material.

In some embodiments of the present disclosure, a top-gate OTFT gas sensor is provided where the top gate electrode is positioned so as not to directly cover, or align with, an active region or channel of the semiconductor layer, so providing gas in the atmosphere an unobstructed pathway to the active region/channel.

In some embodiments, the top gate electrode is patterned, e.g., comprises fingers, comb like structures and/or the like, to provide channels, openings and/or the like that allow passage of the target gas through the top gate electrode to the semiconductor layer.

Organic Semiconducting Layer

Organic semiconductors as described herein may be selected from conjugated non-polymeric semiconductors; polymers comprising conjugated groups in a main chain or in a side group thereof; and carbon semiconductors such as graphene and carbon nanotubes.

An organic semiconductor layer of an OTFT gas sensor as described herein may comprise or consist of a semiconducting polymer and/or a non-polymeric organic semiconductor. The organic semiconductor layer may comprise a blend of a non-polymeric organic semiconductor and a polymer.

Exemplary organic semiconductors are disclosed in WO 2016/001095, the contents of which are incorporated herein by reference.

The organic semiconductor may be crystalline or amorphous. In some embodiments, the organic semiconducting layer of one of the first and second OTFT gas sensors comprises or consists of a crystalline organic semiconductor, optionally a non-polymeric crystalline organic semiconductor, and the organic semiconducting layer of other of the first and second OTFT gas sensors comprises or consists of an amorphous organic semiconductor, optionally a polymeric semiconductor.

The organic semiconducting layer may be deposited by any suitable technique, including evaporation and deposition from a solution comprising or consisting of one or more organic semiconducting materials and at least one solvent. Exemplary solvents include benzenes with one or more alkyl substituents, preferably one or more C₁₋₁₀ alkyl substituents, such as toluene and xylene; tetralin; and chloroform. Solution deposition techniques include coating and printing methods, for example spin coating dip-coating, slot-die coating, ink jet printing, gravure printing, flexographic printing and screen printing.

Optionally, the organic semiconducting layer has a thickness in the range of about 10-200 nm.

Second Semiconducting Layer

In some embodiments the second semiconducting layer, if present, comprises or consists of a transition metal halide or a pseudohalide.

The transition metal halide or pseudohalide may be a metal complex, optionally a coordination polymer. The transition metal of the transition metal halide or pseudohalide is optionally copper (I), silver (I) or cobalt and is preferably Cu (I).

Optionally, the halide of a semiconducting transition metal halide is selected from fluoride, chloride, bromide, iodide or astatide.

Optionally, the pseudohalide of a semiconducting transition metal pseudohalide is selected from thiocyanate, selenocyanate and tellurocyanate.

Preferably, the transition metal halide or pseudohalide is selected from copper thiocyanate (CuSCN); silver thiocyanate (AgSCN); cuprous iodide (CuI). copper selenocyanate (CuSeCN) and copper tellurocyanate (CuTeCN). Copper thiocyanate is particularly preferred.

Blocking Layer

A blocking layer as described herein is preferably a monolayer formed on a surface of the source and drain electrodes of an OTFT gas sensor. A blocking layer may be formed from a binding compound of formula (I):

R—X (I)

where: R is an organic residue and X is a binding group for binding to the surface of the source and drain electrodes. The binding group X may bind to the source and drain electrodes to form a self-assembled monolayer.

X may be selected according to the material of the source and drain electrodes. Preferably, X is a thiol or a silane group. A thiol group X is particularly preferred in the case where the source and drain electrodes are gold.

Preferably, R is a C₁₋₃₀ hydrocarbyl group which may be unsubstituted or substituted with one or more substituents. Exemplary C₁₋₃₀ hydrocarbyl groups are: C₆₋₂₀ aromatic groups, preferably phenyl, phenyl with one or more C₁₋₂₀ alkyl groups; and phenyl C₁₋₂₀ alkyl which may be substituted with one or more C₁₋₂₀ alkyl groups.

Preferred substituents of the C₁₋₃₀ hydrocarbyl group is fluorine and amino, and one or more H atoms of the C₁₋₃₀ hydrocarbyl group may be replaced with fluorine or amino.

Exemplary compounds of formula (I) are:

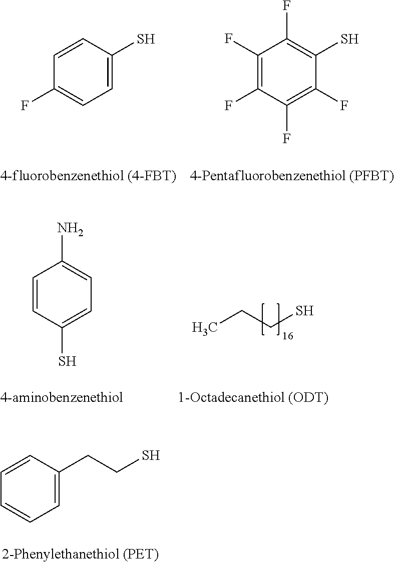

The blocking layer may alter the work function of the source and drain electrodes it is formed on.

The blocking layer may be selected according to the effect, if any, of the blocking layer on the work function of the source and drain electrodes and the required charge injection requirements of the OTFT gas sensor such as the work function—organic semiconductor highest occupied molecular orbital (HOMO) gap in the case of a p-type OTFT gas sensor or the work function—organic semiconductor lowest unoccupied molecular orbital (LUMO) gap in the case of a n-type OTFT gas sensor.

A monolayer may be formed on the source and drain electrodes by depositing the binding compound thereon, for example from a solution of the binding compound in one or more solvents.

The binding compound may be selectively deposited onto the source and drain electrodes only, or may be deposited by a non-selective process such as spin-coating or dip-coating.

A bottom-contact top gate or bottom gate OTFT gas sensor may be formed by depositing the binding compound onto the source and drain electrodes over a dielectric layer and then depositing the organic semiconducting layer. Binding compound which is not bound to the source and drain electrodes, for example binding compound on the dielectric layer following a non-selective deposition process, may be removed by washing.

A gas contacting an electrode surface, such as a gas having a dipole moment, may result in a change in work function at the electrode surface, for example as a result of binding of the gas to the electrode surface. Schottky current dependence on work function may mean that even a relatively small change in work function Δϕ has a large effect on currents J₁ and J₂ at these work functions:

J₂/J₁=e^{-(Δϕ/kT)}

Source and drain electrodes having a blocking layer thereon may undergo a smaller change in work function upon exposure to a gas than the same electrodes without a blocking layer thereon. Optionally, the work function of source and drain electrodes having a blocking layer thereon does not change upon exposure to a gas.

Electrodes

The source and drain electrodes can be selected from a wide range of conducting materials for example a metal (e.g. gold), metal alloy, metal compound (e.g. indium tin oxide) or conductive polymer. The source and drain may be selected according to the material of the blocking layer if one is present.

The gate electrode may be selected from any conducting material, for example a metal (e.g. aluminium), a metal alloy, a conductive metal compound (e.g. a conductive metal oxide such as indium tin oxide) or a conductive polymer.

The length of the channel defined between the source and drain electrodes of the first and second source and drain and gate electrodes of the first and second OTFT gas sensors may be up to 500 microns, preferably less than 200 microns, more preferably less than 100 microns.

Dielectric Layer

The dielectric layer may comprise one or more organic materials, one or more inorganic materials or a mixture thereof. Optionally, dielectric materials are selected from those disclosed in Chem. Rev., 2010, 110 (1), pp 205-239, the contents of which are incorporated herein by reference.

In the case of a top-gate OTFT gas sensor, the dielectric layer preferably comprises an organic material.

Preferred inorganic materials include BaTiO₃, SiTiO₃, SiO₂, SiNx and spin-on-glass (SOG).

Preferred organic materials are organic polymers. Exemplary polymers are, polyvinylpyrrolidine (PVP), acrylates such as polymethylmethacrylate (PMMA) and benzocyclobutanes (BCBs), poly(vinyl cinnamate) P(VCn), and partially fluorinated or perfluorinated polymers, for example poly(vinylidene fluoride-co-hexafluoropropylene) P(VDF-HFP), P(VDF-TrFE-CTFE), and polymers comprising or consisting of tetrafluoroethene repeat units. The polymer may or may not be crosslinked.

In some embodiments, the dielectric layer may consist of a polymer. In some embodiments, the dielectric layer may be a polymer/inorganic composite, for example as described in Materials 2009, 2(4), 1697-1733, the contents of which are incorporated herein by reference. The inorganic material of the composite may be in the form of nanoparticles. The inorganic material of the composite may have a dielectric constant of at least 5, at least 10 or at least 20.

In embodiments, the OTFT gas sensor may comprise more than one dielectric layer, optionally a dielectric bilayer in which a first dielectric layer in direct contact with the organic semiconducting layer comprises a material having a lower dielectric constant than a material of a second dielectric layer spaced apart from the organic semiconducting layer by the first dielectric layer.

In some embodiments, the dielectric layer of a top-gate OTFT gas sensor comprises a gas-permeable material, preferably an organic material, more preferably a polymer material, which allows permeation through the dielectric layer of the gas or gases to be sensed. In some embodiments, the top-gate OTFT gas sensor has a single dielectric layer between the gate electrode and the semiconducting layer. In some embodiments, the top-gate OTFT gas sensor has more than one dielectric layer between the gate electrode and the semiconducting layer, each dielectric layer being permeable to the or each target gas.

The dielectric material may be deposited by thermal evaporation, vacuum processing or lamination techniques as are known in the art. The dielectric material may be deposited from solution using, for example, spin coating or ink jet printing techniques and other solution deposition techniques discussed above with respect to the semiconductor layer. In the case of a bottom gate OTFT gas sensor, the dielectric material should not be dissolved if an organic semiconductor is deposited onto it from solution. In the case of a top-gate OTFT gas sensor, the organic semiconductor layer should not be dissolved if the dielectric is deposited from solution.

Techniques to avoid such dissolution include: use of orthogonal solvents for example use of a solvent for deposition of the organic semiconducting layer that does not dissolve the dielectric layer in the case of a bottom gate device or vice versa in the case of a top gate device; cross linking of the dielectric layer before deposition of the organic semiconductor layer in the case of a bottom gate device; or deposition from solution of a blend of the dielectric material and the organic semiconductor followed by vertical phase separation as disclosed in, for example, L. Qiu, et al., Adv. Mater. 2008, 20, 1141.

The thickness of the dielectric layer is preferably less than 2 micrometres, more preferably less than 500 nm.

Applications

OTFT gas sensors as described herein may be used in detection of alkenes such as conjugated alkene hydrocarbons such as styrene and farnesene or esters such as C₁₋₁₀ alkyl esters, C₁₋₁₀ alkanoate esters, optionally C₁₋₁₀ alkyl-C₁₋₁₀ alkanoate esters, optionally butyl acetate.

A gas sensor system having different OTFTs configured to detect different gases may be used to differentiate between different gases in a gaseous environment.

OTFT gas sensors and gas sensor systems as described herein may be used in an environment in which one or more gases, such as styrene and butyl acetate, are produced by a natural process, e.g. by fruit. The fruit may be apples. Fruit rot may be detected by detection of styrene.

OTFT Gas Sensor 1

A PEN substrate was baked in a vacuum oven and then UV-ozone treated for 30 seconds. Source and drain contacts were deposited onto the substrate by thermal evaporation of 3 nm Cr followed by 40 nm Au through shadow masks with channel length of 125 μm and a channel width of 4 mm. A solution of 4-aminobenzenethiol and 4-fluorobenzenthiol (1:1) was spin-coated onto the source and drain electrodes from an isopropyl alcohol solution (0.1% v/v). After 2 minutes, the substrate and source and drain electrodes were rinsed with isopropyl alcohol. Semiconducting Polymer 1, illustrated below, was deposited over the substrate by spin coating from a 1% w/v solution in 1,2,4-trimethylbenzene to a thickness of 40 nm and dried at 100° C. for 1 or 10 min in air. The polymer dielectric Teflon® AF2400 was spin coated from a 2.5% w/v solution in a 50:50 v/v blend of fluorinated solvents FC43 and FC85 to a 300 nm thickness and dried at 80° C. for 10 min, after a 5 minute initial drying phase while spinning. The gate was formed by thermal evaporation of Cr (3 nm) followed by Al (200 nm) through a shadow mask to form a gate electrode having a comb structure with comb fingers of 125 microns width and gaps of 125 microns between fingers.

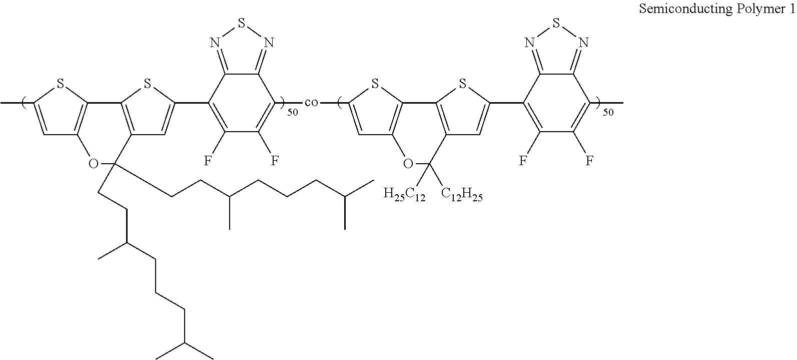

OTFT Gas Sensor 2

A PEN substrate was dehydration baked in a vacuum oven. A bottom gate electrode was deposited by thermal evaporation of 3 nm of Cr, followed by evaporation of 40 nm of Al. A dielectric layer was formed by spin-coating a pre-polymer solution of poly-vinyl cinnamate from a 4% solution in chlorobenzene to a thickness of 200 nm followed by a 20 minute UV cure and then drying at 100° C. for 1 hour. An organic semiconducting layer was formed by spin-coating a 1% w/v solution of 3:1 blend of 6,13-Bis(triisopropylsilylethynyl)pentacene (TIPS-pentacene) and polystyrene (MW=650 kDa) in 1,2,4-trimethylbenzene to a thickness of ˜40 nm followed by drying for 10 minutes at 100° C. Source and drain contacts were deposited by thermal evaporation of 40 nm Au through shadow masks with channel length of 125 μm and channel width of 4 mm.

A comparison of OTFT Gas Sensors 1 and 2 is set out in Table 1

TABLE 1

| | | Thiol | Organic |
|---|---|---|---|
| | Device type | layer | semiconductor |
| OTFT Gas Sensor 1 | Top gate, bottom | Y | Amorphous |
| | contact | | |
| OTFT Gas Sensor 2 | Bottom gate, top | N | Crystalline |
| | contact | | |

The OTFT gas sensors were pulsed for 100 ms every 25 s at a drain and gate voltage of −4V to give a sensor current of >10 nA.

OTFT Gas Sensors 1 and 2 were exposed to 0.1 ppm of styrene for 1 hour.

OTFT Gas Sensors 1 and 2 were exposed to 4,000 ppm of butyl acetate for 1 hour

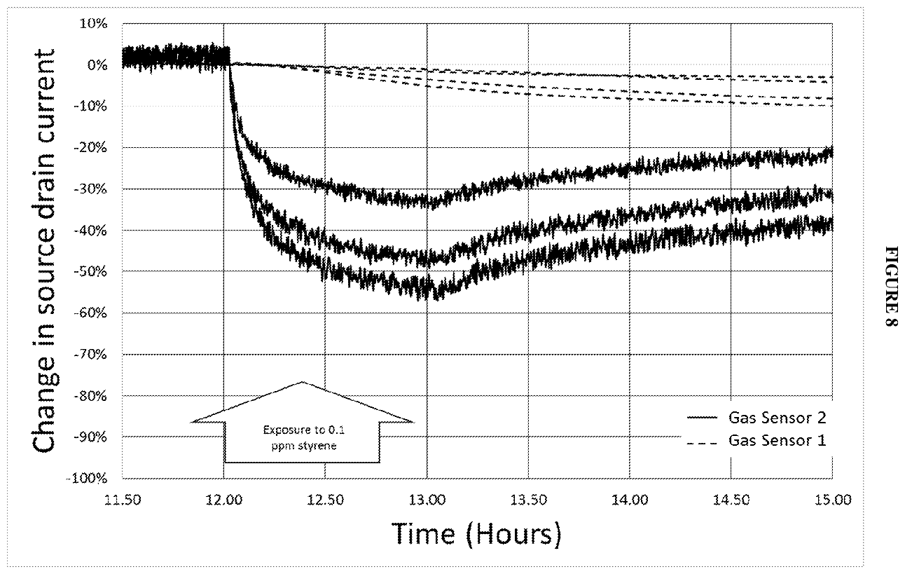

With reference to FIG. 7, OTFT Gas Sensor 1 is much more sensitive to butyl acetate than OTFT Gas Sensor 2. Without wishing to be bound by any theory, the crystalline TIPS-pentacene of OTFT Gas Sensor 2 is less permeable than the amorphous semiconducting polymer of OTFT Gas Sensor 1.

Conversely, and with reference to FIG. 8, OTFT Gas Sensor 2 is much more sensitive to styrene than Gas Sensor 1. Without wishing to be bound by any theory, styrene is able to bind to the source and drain electrodes of Gas Sensor 2 but the thiol layer of Gas Sensor 1 inhibits such binding.

1. A gas sensor system configured to detect a conjugated hydrocarbon in an environment, comprising:
a first organic thin film transistor (OTFT) gas sensor comprising:
first source and drain electrodes in electrical contact with a first organic semiconductor layer;
a first gate electrode; and
a first gate dielectric disposed between the organic semiconductor layer and the gate electrode, wherein the first OTFT gas sensor is configured to provide for gas communication of the conjugated hydrocarbon with the first source and drain electrodes; and
a second OTFT gas sensor comprising:
second source and drain electrodes in electrical contact with a second organic semiconductor layer;
a second gate electrode; and
a second gate dielectric disposed between the second organic semiconductor layer and the second gate electrode, wherein the second OTFT gas sensor is configured to block gas communication of the conjugated hydrocarbon with the second source and drain electrodes.

2. A gas sensor system according to claim 1, wherein the first organic semiconductor layer is in direct contact with the first source and drain electrodes.

3. A gas sensor system according to claim 1, wherein the first organic semiconductor layer comprises a crystalline organic semiconductor.

4. A gas sensor system according to claim 1, wherein a blocking layer is disposed on a surface of the second source and drain electrodes between the second source and drain electrodes and the second organic semiconducting layer.

5. A gas sensor system according to claim 4, wherein the blocking layer is a monolayer.

6. A gas sensor system according to claim 5, wherein the blocking layer is a thiol.

7. A gas sensor system according to claim 1, wherein the second organic semiconductor layer of the second OTFT gas sensor comprises an amorphous organic semiconductor.

8. A gas sensor system according to claim 1, wherein the second OTFT gas sensor is configured to detect an ester.

9. A gas sensor system according to claim 8 wherein the ester is a C₁₋₁₀ alkyl ester.

10. A gas sensor system according to claim 8 wherein the ester is a C₁₋₁₀ alkanoate ester.

11. A gas sensor system according to claim 1 wherein the conjugated hydrocarbon comprises a conjugated alkene group.

12. A gas sensor system according to claim 1 wherein the conjugated hydrocarbon is styrene.

13. A method of identifying the presence and/or concentration of a conjugated hydrocarbon and/or an ester in an environment, comprising:
measuring a response of first and second OTFT gas sensors, according to claim 1, to the environment; and
determining from the measured responses a presence and/or a concentration of the conjugated hydrocarbon and/or the ester.

14. An organic thin film transistor (OTFT) gas sensor configured to detect a conjugated hydrocarbon in an environment, comprising:
source and drain electrodes in electrical contact with an organic semiconductor layer;
a gate electrode; and
a gate dielectric between the organic semiconductor layer and the gate electrode, wherein the OTFT gas sensor is configured to provide for gas communication of the conjugated hydrocarbon in the environment with the source and drain electrodes.

15. A method of identifying the presence and/or concentration of a conjugated hydrocarbon in an environment, comprising:
measuring of a response of an OTFT gas sensor according to claim 14 to the environment; and
determining from the measured response a presence and/or a concentration of the conjugated hydrocarbon.

16. An organic thin film transistor (OTFT) gas sensor configured to detect an ester in an environment, comprising:
source and drain electrodes in electrical contact with an organic semiconductor layer;
a gate electrode; and
a gate dielectric between the organic semiconductor layer and the gate electrode, wherein the OTFT gas sensor is configured to block gas communication of gases in the environment with the source and drain electrodes.

17. A method of identifying the presence and/or concentration of an ester in an environment, comprising:
measuring a response of an OTFT according to claim 16 to the environment; and
determining from the measured response presence and/or a concentration of the ester.
